(19) United States
(12) Patent Application Publication
(10) Pub. No.: US 2003/0167047 A1
(43) Pub. Date: Sep. 4, 2003

(54) Pad for absorbing bodily fluid

(76) Inventor: Nini Policappelli, Los Angeles, CA (US)

Correspondence Address: GREENBERG TRAURIG LLP, 2450 COLORADO AVENUE, SUITE 400E, SANTA MONICA, CA, 90404, US

(21) Appl. No.: 10/373,563

(22) Filed: Feb. 24, 2003

Related U.S. Application Data
(60) Provisional application No. 60/360,570, filed on Mar. 1, 2002.

(51) Int. Cl. A61F 13/15

(52) U.S. Cl. 604/385.01

(57) ABSTRACT

A sanitary pad includes an area of absorptive material and an area of nonabsorptive or less absorptive material. This non-absorptive or less absorptive area is ideally located centrally in the pad for location adjacent the mouth of the vagina. This area is optionally inflatable between a retracted position and an extended position.

### BACKGROUND OF THE INVENTION

1. Field of the Invention

This invention relates to a pad for absorbing bodily fluid. In particular, the invention is concerned with sanitary pads.

Different shapes and forms of sanitary pads are known. In particular, different constructions are known to facilitate the comfortable location of pads around the vaginal area. This location often is not as comfortable as it should be, and the means for locating the pad, namely, suitable adhesive strips doesn't necessarily provide for effective securement. Difficulties often arise with the relative amount of fluid to be absorbed and the size of the pad.

This invention is directed to overcoming the drawbacks of known sanitary pads, and to minimize infection and toxic shock syndrome.

### SUMMARY OF THE INVENTION

A pad for absorbing bodily fluid and for location adjacent to and outside the vaginal area, comprises an absorptive planar pad for location over the mouth of the vagina and peripherally about the mouth so that fluid from the vagina can be absorbed by the absorptive pad. A central area of the absorptive pad is located over the mouth and the central area selectively has less absorptive material wherein more fluid is absorbed and the pad area removed from the mouth.

The central area preferably has no absorptive material. The central area includes a raised element and the area around the raised element is surrounded by absorptive material. The raised area is movable between a retracted hand and extended position and the raising hand protraction of the raised area is effected under the action of an inflatable element.

The inflatable element includes a valve and a pump and the pump includes a flexible balloon element to permit the passage of a fluid from one side of the valve to the other side and thereby permit inflation or deflation of the element.

In different forms of the pad, one or more non-absorptive elements are located around the periphery of the pad. In one or more cases, the non-absorptive elements are adhesively attached to the pad.

On an opposite side of the absorptive element, there can be selectively one or more adhesive elements, the adhesive elements being located preferably towards the periphery of the pad.

The absorptive element includes multiple layers of absorptive material and each layer of the absorptive material having different characteristics of absorption.

### BRIEF DESCRIPTION OF THE DRAWINGS

FIG. 1 is a plan view of the sanitary pad when viewed from the side for location adjacent the vaginal area.

FIG. 2 is a side view of the pad in an exploded form.

FIG. 3 is an end view of the pad from the one end.

FIG. 4 is a cross-section end view of the pad along line 44.

FIG. 5 is a side view of the central element of the pad without the absorptive materials showing the inflatable member in the deflated position.

FIG. 6 is an opposite side view of the central portion of the pad showing the inflatable member in the inflated position.

FIG. 7 is a plan view of a different cross-sectional shape of a pad.

FIG. 8 is a side view exploded to show the different proponents of the pad of FIG. 7.

FIG. 9 is an opposite plan view of the pad.

FIG.10 is a cross-sectional view of the pad along line 10-10 of FIG. 7.

FIG. 12 is a side view of the pad with the inflatable member non-inflated.

FIG. 13 is a side view of the central element of the pad with the inflatable member inflated.

### DETAILED DESCRIPTION OF THE PREFERRED EMBODIMENTS

In the following description of the preferred embodiments reference is made to the accompanying drawings which form the part thereof, and in which are shown by way of illustration of specific embodiments in which the invention may be practiced. It is to be understood that other embodiments may be utilized and structural and functional changes may be made without departing from the scope of the present invention.

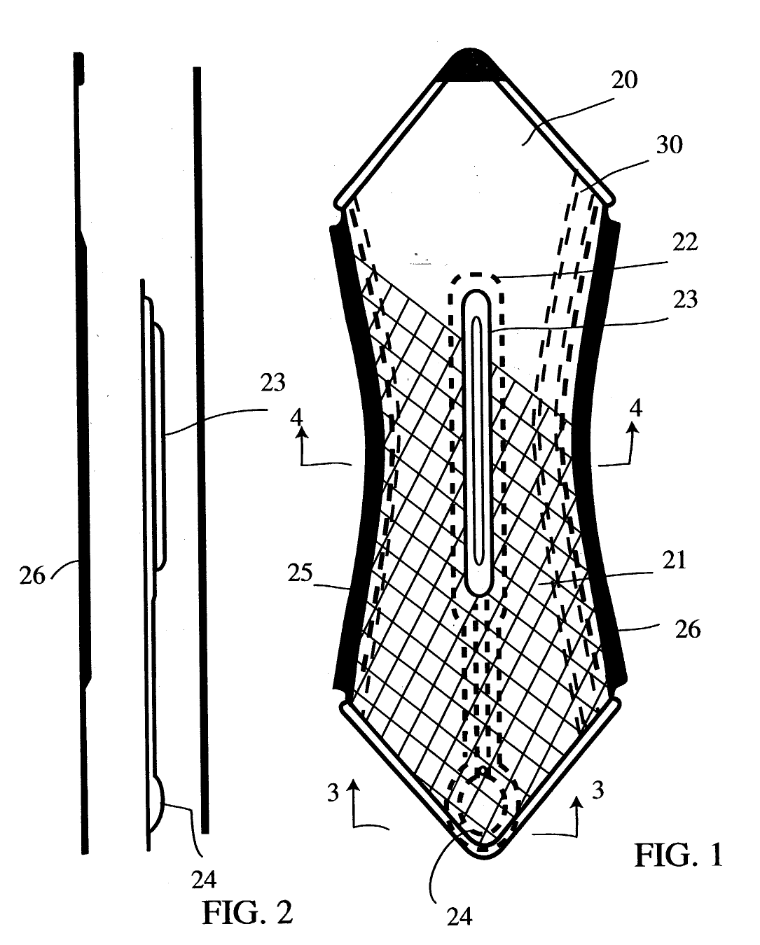

In FIG. 1 there is shown a plan view of a pad with a surface 20 for engagement over the vaginal entrance.

Multiple different layers of cotton or absorptive material 21 are provided on the surface to absorb bodily fluid from the vaginal area. The central area 22 has less or no absorptive material and includes an inflatable member 23. The purpose of this member is to deflect bodily fluid from the central area where it egresses from the vagina to the peripheral areas above the central area. The central portion 23 may or may not be inflatable and may or may not have absorptive characteristics.

A pump 24 is provided at one end and has a fluid connection with the central area 23 so that in the embodiment where the area 23 is inflatable, the pump can force air into the inflatable area to extend, as required, and rise to a level substantially at the height of the planar absorptive material, above the absorptive material planar surface or below that surface.

At the periphery of the surface 20, there is provided one or more silicon-like non-absorptive elements 25 and 26 which can be provided with a certain degree of adhesiveness or stickiness. These materials can abut against the body and provide for better comfort when locating the pad in adjacency with the body, and avoid irritation.

As illustrated in FIG. 2, the pad can be formed of three layers. One or more absorptive or base pads to either side of the central area which can be the selectively inflatable area to rise centrally in the pad.

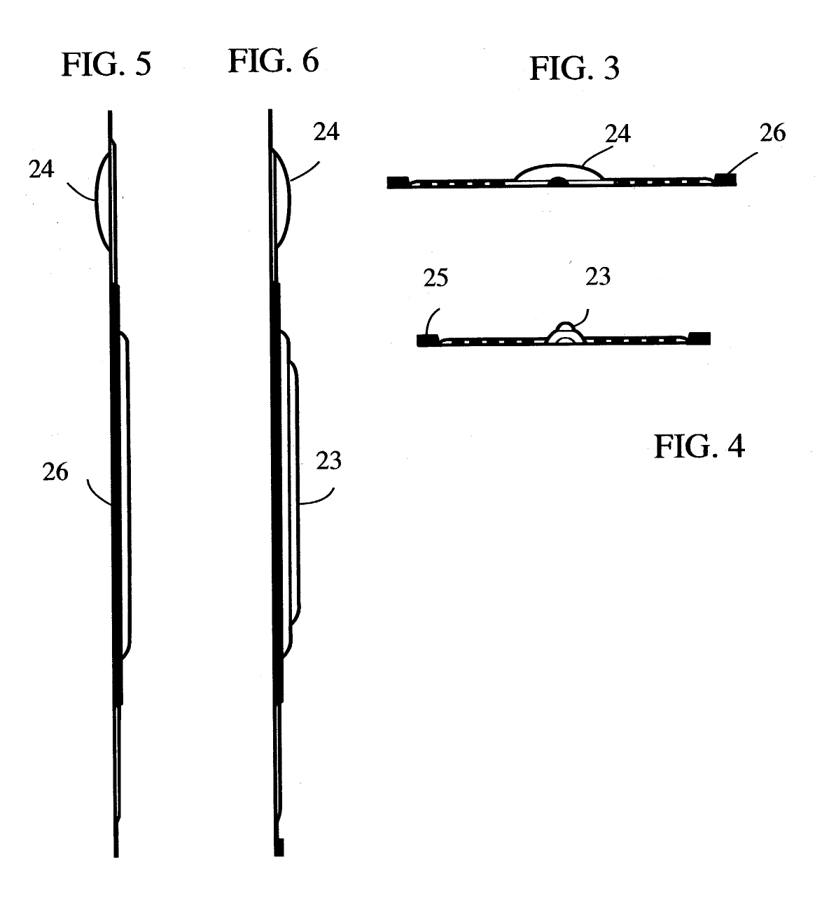

FIG. 3 shows the balloon pump 24 in cross section which is used to inflate the inflatable element 23 as illustrated in FIG. 4. One or more valves can be located between the balloon area and the inflatable area 23 so that fluid pumped from the pump 24 stays within the inflatable area 23.

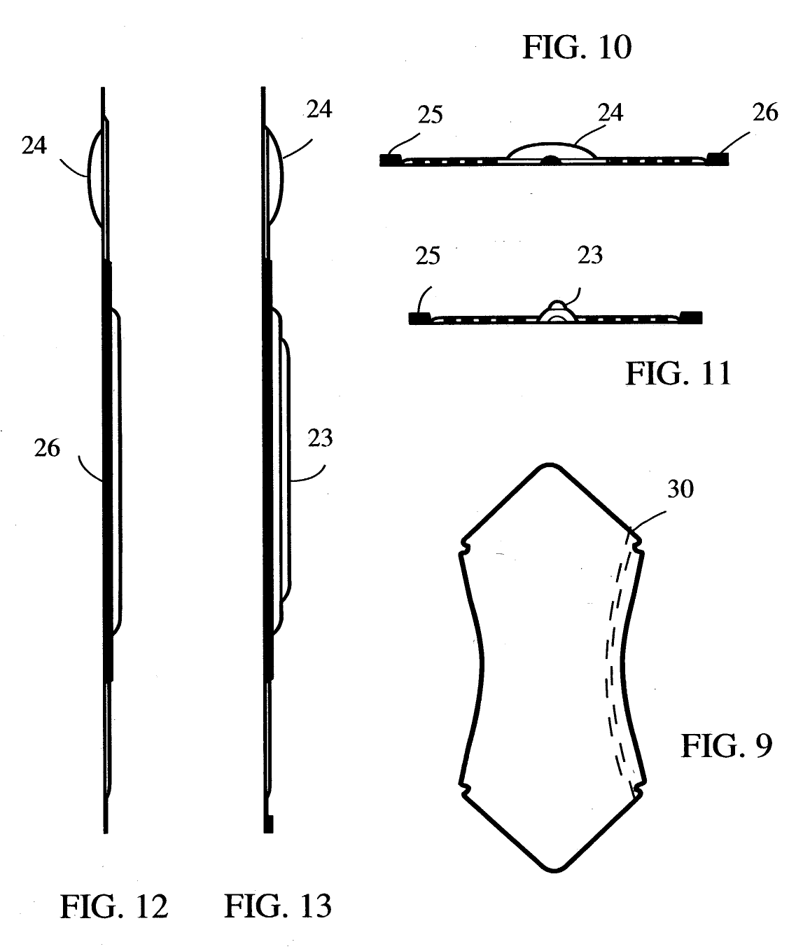

As seen in FIG. 9 there is a different planar view to the pad per the same characteristics are provided in the central area such that the central area has less, and sometimes no, absorptive material and thereby causes the fluid from the central area to move towards the peripheral areas. This provides for increased comfort for the user of the pad.

Many other forms of the invention exist, each different from the others in matters of detail only.

On the side opposite the area from which there is a protrusion, there can be provided one or more adhesive elements to prevent the pad to adhere to the inside surface of the inner garments of the wearer. One or more other adhesive members can be provided on the opposite side if desired.

In some variations there can be a situation without a valve. Other variations can be that existing type pad wherein the central area raised and/or non-absorbative element can be applied to the zone which is a central zone of the pad.

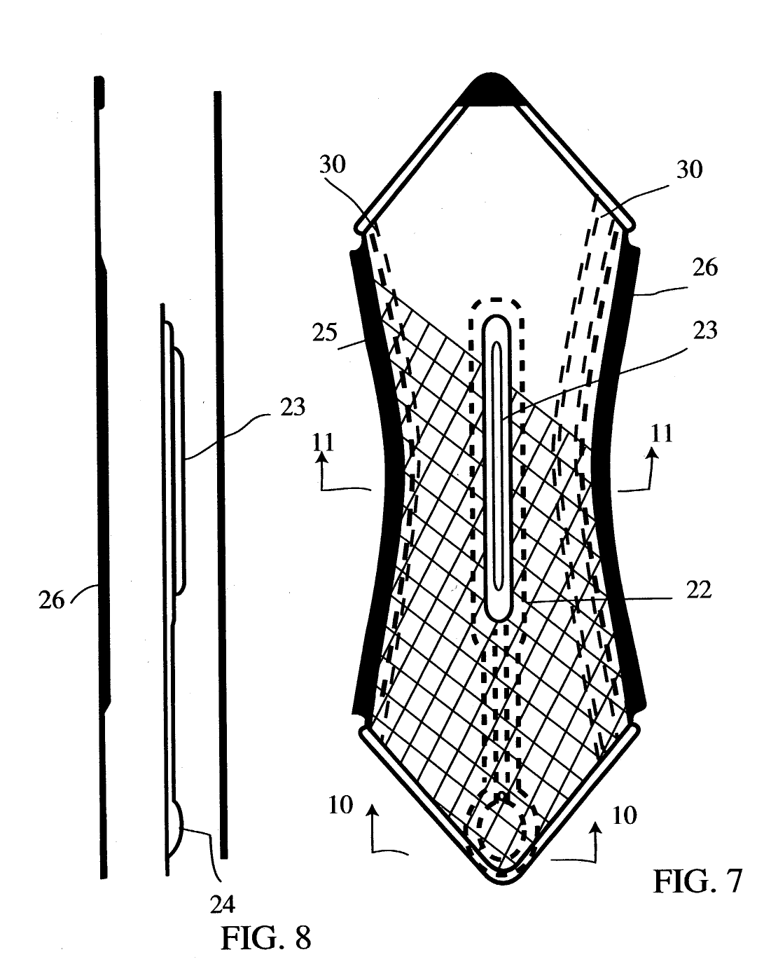

In the pad there can be a structure where there are foldable zones in the pad. Thus there is a pad which can have a planar area which can be folded into a smaller size to fit a smaller area, or can be expanded to fit a larger area. The fold/crease/pleat zones are shown as 30. On the right hand side of FIGS. 1 and 7 this is shown expanded on the left hand side this is shown contracted. There may also be cases where there is no pump. In other variations the non-absorbative and/or zone may be off-center. It may have different shapes and sizes.

Many modifications and variations are possible in the light of the above teaching. The foregoing is a description of the preferred embodiments of the invention and has been presented for the purpose of illustration and description. It is not intended to be exhaustive and so limit the invention to the precise form disclosed.

The internal area 23 can be a balloon like element which may be at least partly filled with one or more of a liquid and /or a resin and/or a sponge material. The area can be in a normally relatively dry state and the material may be vacuum packed in the central area. In use, the vacuum package is opened so that it becomes open to the environment and thereby expandable. Such expansion is promoted when the area 23 becomes moist so it can extend or expand.

The invention is to be determined by the following claims

What is claimed is:

1. A pad for absorbing bodily fluid and for location adjacent to and outside the vaginal area comprising:
an absorptive planar pad for location over the mouth of the vagina and peripherally above the mouth so that fluid from the vagina area can be absorbed by the absorptive pad; and
a central area of the absorptive pad being for location over the mouth of the vagina, the central area selectively having less, and selective no, absorptive material wherein more fluid is absorbed in the pad area removed from the mouth.

2. A pad as claimed in claim 1 wherein the central area has no absorptive material.

3. A pad as claimed in claim 1 wherein the central area includes a raised element, the end of the raised element being surrounded by absorptive material.

4. A pad as claimed in claim 1 wherein the raised area is movable between a retracted and an extended position.

5. A pad as claimed in claim 1 wherein the raised area is raised under the action of an inflatable element.

6. A pad as claimed in claim 5 wherein the inflatable element includes a valve and a pump, the pump including a flexible element to permit the passage of a fluid from one side of the valve to the other and thereby permit inflation or deflation of the element.

7. A pad as claimed in claim 1 including non-absorptive elements around the periphery of the pad.

8. A pad as claimed in claim 1 including a silicone material selectively around selected portions of the edge of the pad.
